(19) United States
(12) Patent Application Publication
(10) Pub. No.: US 2003/0059930 A1
(43) Pub. Date: Mar. 27, 2003

(54) Automated device for the processing, signal acquisition and analysis of biochips

(75) Inventor: Michel Gazeau, Saint-Julien-en-Genevois (FR)

Correspondence Address: SCHNADER HARRISON SEGAL & LEWIS, LLP, 1600 MARKET STREET, SUITE 3600, PHILADELPHIA, PA, 19103

(73) Assignee: Genomic S.A.

(21) Appl. No.: 10/238,863

(22) Filed: Sep. 9, 2002

Related U.S. Application Data
(63) Continuation of application No. PCT/FR01/00717, filed on Mar. 9, 2001.

(30) Foreign Application Priority Data
Mar. 9, 2000 (FR) 00/03139

(51) Int. Cl. C12M 1/34

(52) U.S. Cl. 435/287.2

(57) ABSTRACT

A reactor for biological analysis of biochips comprising a body and means for moving the biochip in relation to a surface of the body between a first position in which the biochip is close to the surface to define a first volume and a second position in which the biochip is moved away from the surface to define a second dynamically variable volume.

### RELATED APPLICATIONS

This is a continuation of International Application No. PCT/FR01/00717, with an international filing date of Mar. 9, 2001, which is based on French Patent Application No. 00/03139, filed Mar. 9, 2000.

1. Field of the Invention

This invention relates to an automated device for processing, signal acquisition and analysis of biochips, particularly to a reactor for such biological analysis.

2. Background

Biochips enable parallel analysis of a very large number of molecules, essentially nucleic acids and proteins. The basic principle is recognition and pairing of two molecules that have affinities. One of the collections of molecules is fixed in the form of minideposits or microdeposits on a solid support, fabric, glass slide, silicon chip, etc.

The other molecule, which is labeled and generally in solution, is brought into contact with the samples deposited on the solid support. After an incubation period, the excess of labeled molecule is eliminated and the support is carefully washed. It is then necessary to detect and quantify the signal emitted by the molecules retained on the deposits. In certain cases, the retained molecule can be uncoupled from the deposits and a new molecule can be tested with the same solid support.

It is generally necessary to bring the deposits into contact with different reagents and then to wash them carefully to process biochips. It is necessary to adjust the temperature of the reagents and the biochips. The most frequently employed tags are fluorescent, but other labeling techniques can be used.

The labeled molecule is a rare and/or expensive element. It is desirable to minimize the volume required. The other reagents, particularly the washing products, are not expensive and the reduction of the volumes used is much less important.

### SUMMARY OF THE INVENTION

This invention relates to a reactor for biological analysis of biochips including a body and means for moving the biochip in relation to a surface of the body between a first position in which the biochip is close to the surface to define a first volume and a second position in which the biochip is moved away from the surface to define a second dynamically variable volume.

### BRIEF DESCRIPTION OF THE DRAWINGS

Better understanding of the invention will be obtained from the description below, provided for purely explanatory purposes, of one mode of implementation of the invention with reference to the attached figures:

FIGS. 1, 2 and 3 illustrate a front view, a side view and a top view of the reactor, respectively;

FIG. 4 illustrates a top view of the centrifuge;

FIGS. 5 and 6 illustrate a side view and a front view of the centripetal valve carrier device, respectively;

FIGS. 7, 8 and 9 illustrate a side view, a front view and a top view of the centrifuge drainage carrier device, respectively; and

FIGS. 10 and 11 illustrate a side view and a front view of the capillary carrier device, respectively.

### DETAILED DESCRIPTION

It will be appreciated that the following description is intended to refer to specific embodiments of the invention selected for illustration in the drawings and is not intended to define or limit the invention, other than in the appended claims.

This invention pertains to an automated device capable of processing biochips without human intervention and acquiring signals for their subsequent analysis. The automated device enables processing of a large number of biochips in addition to cost savings in terms of reagents. The equipment is relatively inexpensive because of its simplicity.

An important aspect of the device is an assembly of reactors which are described below.

The cover of the centrifuge comprises various elements:

A number of fixed nozzles spaced apart in a regular manner equal to the number of reactors of the rotor and located to dispense liquid into the reactors. These nozzles are connected by calibrated catheters to the cover of a hermetic enclosure which comprises a number of tubes containing the labeled molecules. This enclosure can be subjected to a selected pressure for a selected period of time by means, e.g., of a gas carboy, which has the effect of flowing from the catheters to the reactors a substantially equal volume of the liquids contained in the tube. This enclosure can be cooled by any process to ensure good conservation of the labeled molecules.

A number of nozzles affixed on the cover between the preceding ones, connected to bottles under pressure or under reduced pressure, or to pumps, allows successive distribution in each reactor of the common reagents in large volume (about several milliliters). Certain of these nozzles can discharge liquids for cleaning and decontamination of the device.

A mechanical translational movement device makes it possible to push away then to bring back to its initial position a gliding biochip-carrier part from each reactor or in other cases the reactors themselves, successively.

The temperature inside the enclosure containing the reactors is regulated. For this purpose, the mobile blade comprises a thermostating element for heating and/or cooling the biochip. These heating or cooling elements are operated by a Peltier effect element. They can also be implemented by conduits for the circulation of coolant fluids making it possible to adjust the temperature cycles between about 97° C. and about 50° C. with cyclical temperature changes. Thermoregulation can also be implemented by a capillary pump two phase loop. If necessary, the hygrometry of the air in the enclosure can be controlled.

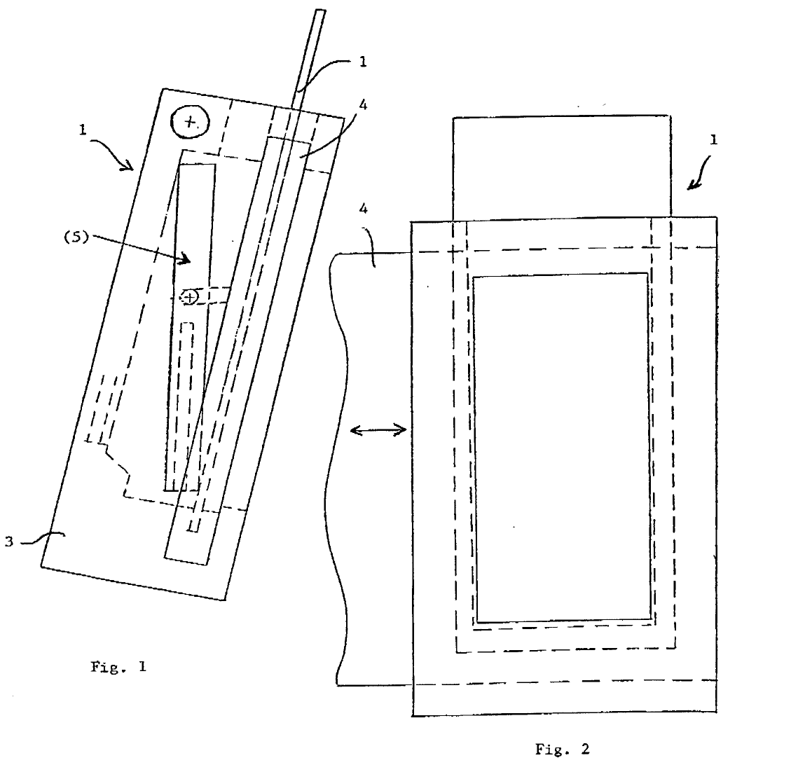

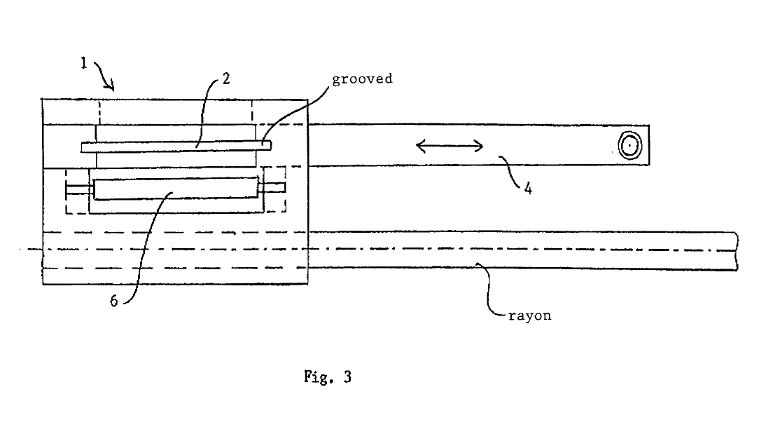

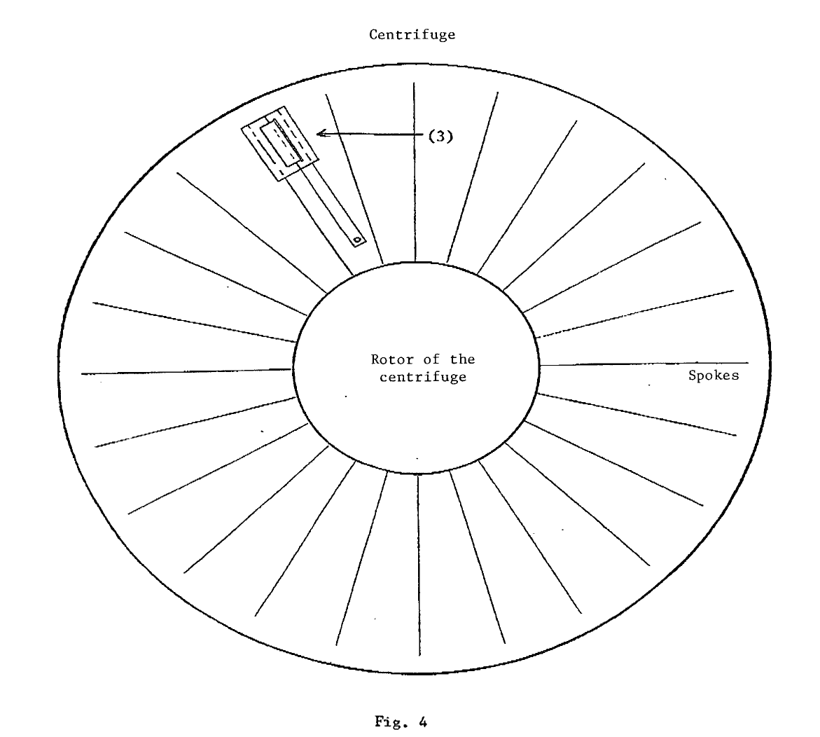

Turning now to the drawings, the invention concerns a reactor (1), illustrated in FIGS. 1 to 3, for biological analysis by biochips (2), wherein the biochip is mobile in relation to a surface facing it between a first position in which the biochip (2) is close to a surface to define a first volume of small content and a second position in which the biochip is moved away from said surface to define a dynamically variable volume.

The reactors (1) are aligned and inclined such that the biochips (2) can receive the dispensed reagents directly. They are constituted of three elements:

1) The body (3) of the reactor. It has a track on one of its two large surfaces. It can also have on one of its perpendicular surfaces an opening blocked by a stopper from the interior and held in position by a spring. This opening is connected to a pipeline allowing injection of a liquid under pressure into the reactor.

2) A biochip-carrier sliding part (4), which when it is provided with a biochip and engaged at the bottom in its track, blocks one of the two large surfaces of the reactor. The biochip carrier can be moved by about one or about two millimeters in its track, without harming the tightness of the reactor to improve the contact of the deposits with the reagents. It can be moved away from its original position towards the exterior by about six or about seven millimeters to open the reactor and allow the discharge of its content. Finally, it can be moved away from its original position by about several centimeters to be brought out of the centrifuge enclosure and placed in the field of the signal acquisition system. The biochip carriers or, in other cases, the reactors are activated by the mechanical translational movement device mentioned above.

3) An agitator (5) composed of a blade (6) which can either be supported on the lower part of the biochip (2) forming a solid angle which retains a small volume of liquid, or from this position come to be applied on the biochip spreading over its surface and without air bubble the volume of liquid retained, or enter completely into the body of the reactor to allow the sliding of the biochip carrier.

The continuous passage between these three positions causes the discharge of the washing liquid on the active surface of the biochip.

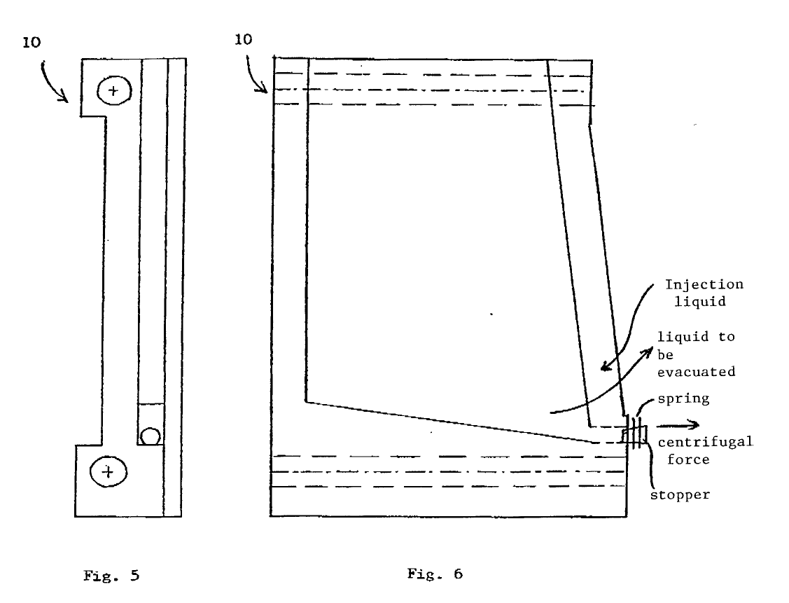

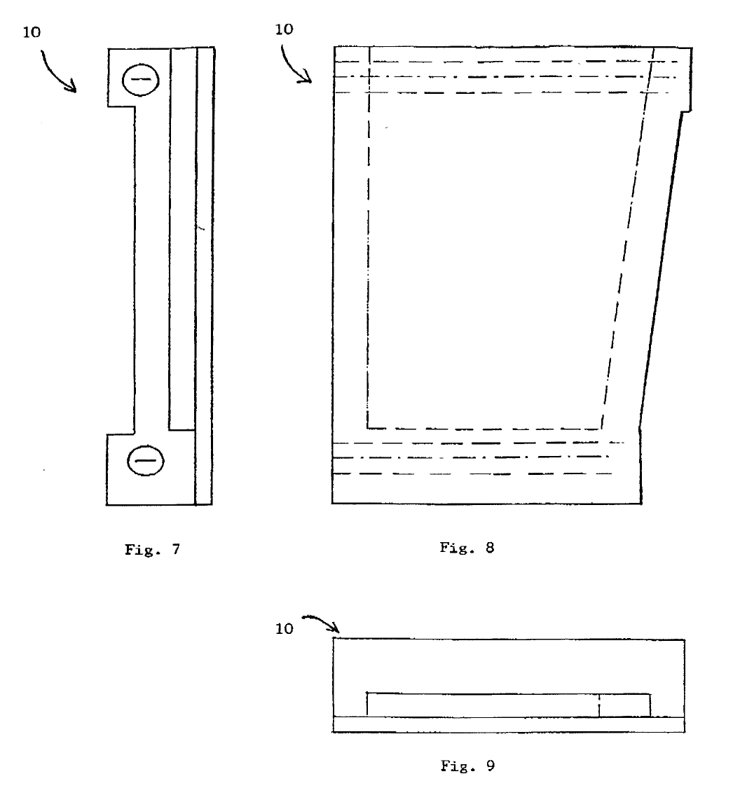

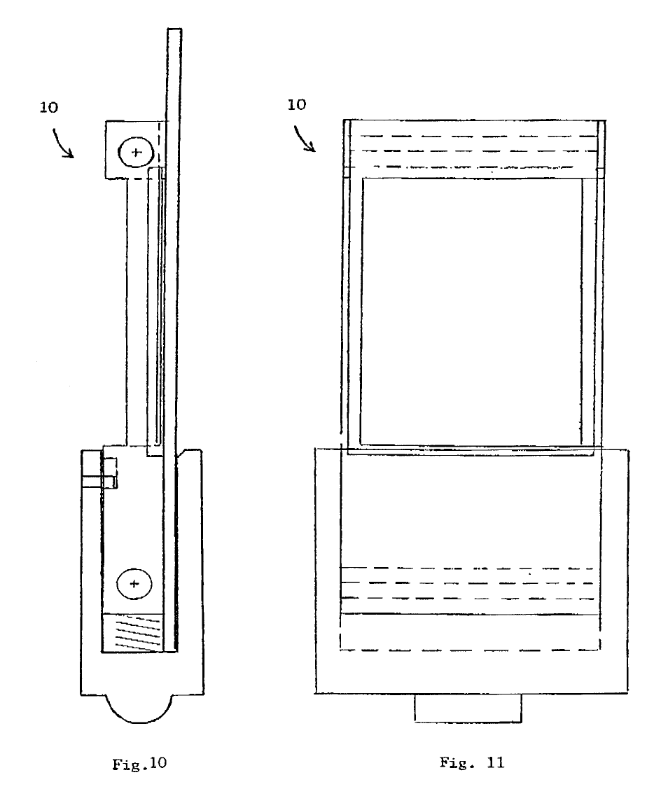

Other types of reactors can be envisaged such as, for example, the three versions of the carrier devices (10) presented in FIGS. 5 and 6, 7 to 9 and 10 and 11. They are simpler and less expensive to manufacture, but they do not allow approaching very close to the active surface of the biochip for acquisition of the signal. In this case it is the entire reactor which is pushed to the exterior of the centrifuge's enclosure.

The biochip is formed from a glass strip of the microscope slide type on which a frame of hydrophobic paint about two to about three millimeters in width and regular in thickness was deposited. One of the sides ofthis frame is interrupted to allow passage of the liquids to the bottom of the biochip.

1. A reactor for biological analysis of biochips comprising:
a body and means for moving the biochip in relation to a surface of the body between a first position in which the biochip is close to the surface to define a first volume and a second position in which the biochip is moved away from the surface to define a second dynamically variable volume.

2. The reactor according to claim 1, wherein the means for moving the biochip is a sliding biochip-support which, when provided with the biochip and engaged in the bottom of a track, blocks one of two surfaces of the reactor.

3. The reactor according to claim 1, wherein the body has an aperture for introduction of a sliding part and an agitator into the body.

4. The reactor according to claim 3, wherein the body has on one of its two surfaces a track for the sliding part.

5. The reactor according to claim 3, wherein the body has a perpendicular surface with an opening blocked by a stopper located in an interior portion of the body and held in a selected position by a spring.

6. The reactor according to claim 5, wherein the opening is connected to a pipeline which enables injection of a liquid under pressure into the reactor.

7. The reactor according to claim 2, wherein the sliding biochip support blocks one of two surfaces of the reactor when the biochip support piece is provided with the biochip and engaged in a bottom portion of the track.

8. The reactor according to claim 2, wherein the biochip support can be moved by about one to about two millimeters in the track to improve contact of deposits with reagents.

9. The reactor according to claim 2, wherein the biochip support can be moved away from one selected position toward an exterior portion of the body by about six to about seven millimeters to open the reactor and allow outward flow of fluid contents therein.

10. The reactor according to claim 2, wherein the biochip support can be moved away from one selected position by about several centimeters to be brought outwardly of the body and placed in a field of a signal acquisition system.

11. The reactor according to claim 3, wherein the agitator is composed of a blade which can either be 1) supported on a lower part of the biochip forming an angle which retains a small volume of liquid, or be applied on the biochip to spread over a surface of the biochip and without forming an air bubble the volume of liquid retained, or 2) enter completely into the body of the reactor to allow sliding of the biochip support.
